(19) United States
(12) Patent Application Publication
(10) Pub. No.: US 2022/0207060 A1
(43) Pub. Date: Jun. 30, 2022

(54) INFORMATION PROCESSING DEVICE AND INFORMATION PROCESSING PROGRAM

(71) Applicant: KANEKA CORPORATION, Osaka (JP)

(72) Inventor: Yoshiyuki NASUNO, Osaka-shi (JP)

(73) Assignee: KANEKA CORPORATION, Osaka (JP)

(21) Appl. No.: 17/655,167

(22) Filed: Mar. 16, 2022

Related U.S. Application Data
(63) Continuation of application No. PCT/JP2020/028061, filed on Jul. 20, 2020.

(30) Foreign Application Priority Data
Sep. 19, 2019 (JP) 2019-170817

(51) Int. Cl. G06F 16/28; G06F 16/2457

(52) U.S. Cl. CPC G06F 16/285; G06F 16/24575

(57) ABSTRACT

A matching device comprises an answer acquisition unit, a biometric information acquisition unit, an assessment unit, a classification unit, and a matching unit. The answer acquisition unit acquires an answer from a subject person to a question that is addressed to the subject person for matching the subject person with other persons. The biometric information acquisition unit acquires the biometric information of the subject person. The assessment unit assesses the state of the subject person on the basis of the biometric information acquired by the biometric information acquisition unit. The classification unit performs classification on the basis of the answer acquired by the answer acquisition unit and the state of the subject person assessed by the assessment unit in order to achieve matching of the subject person. The matching unit matches the subject person with other persons on the basis of the classification result of the classification unit.

### CROSS-REFERENCE TO RELATED APPLICATIONS

This application claims benefit of priority to International Patent Application No. PCT/JP2020/028061, filed Jul. 20, 2020, and to Japanese Patent Application No. 2019-170817, filed Sep. 19, 2019, the entire contents of each are incorporated herein by reference.

### BACKGROUND

### Technical Field

The present disclosure relates to an information processing device and an information processing program.

### Background Art

In recent years, appropriate matching based on users' characteristics and preferences has been demanded in various fields. For example, appropriate matching has been demanded in a field for making a match between users seeking a personal relationship and a field for making a match between a user providing a predetermined service and a user utilizing the predetermined service.

One example of the technique for such matching between the users is disclosed in Japanese Unexamined Patent Application, Publication No. 2019-062460. The technique disclosed in Japanese Unexamined Patent Application, Publication No. 2019-062460 is intended to make a match between a client who makes a call and an operator responding to such a call at a call center. Thus, in the technique disclosed in Japanese Unexamined Patent Application, Publication No. 2019-062460, the state (e.g., the level of tiredness) of the operator is specified based on biometric information on the operator. Then, the client is matched with the appropriate operator based on the specified state of the operator and the level of difficulty in the contents of an inquiry from the client.

### SUMMARY

A typical technique such as the technique disclosed in Japanese Unexamined Patent Application, Publication No. 2019-062460 above is utilized so that a match can be made. However, it has been demanded that users are more appropriately matched with each other not only based on the biometric information as in the conventional technology but also from various points of view.

The present disclosure has been made in view of such a situation. Accordingly, the present disclosure provides an information processing device and an information processing program for more appropriately matching users with each other from various points of view.

The information processing device according to the present disclosure includes a response acquisition unit that acquires a response from a subject person to a question provided to the subject person for matching the subject person with any other person, a biometric information acquisition unit that acquires biometric information on the subject person, and a determination unit that determines the state of the subject person based on the biometric information acquired by the biometric information acquisition unit. The information processing device further includes a classification unit that classifies the subject person for matching based on the response acquired by the response acquisition unit and the state of the subject person determined by the determination unit, and a matching unit that matches the subject person and the other person with each other based on a classification result obtained by the classification unit.

The information processing program according to the present disclosure causes a computer to implement the response acquisition function of acquiring a response from a subject person to a question provided to the subject person for matching the subject person with any other person, the biometric information acquisition function of acquiring biometric information on the subject person, the determination function of determining the state of the subject person based on the biometric information acquired by the biometric information acquisition function, the classification function of classifying the subject person for matching based on the response acquired by the response acquisition function and the state of the subject person determined by the determination function, and the matching function of matching the subject person and the other person with each other based on a classification result obtained by the classification unit.

According to the present disclosure, the users can be more appropriately matched with each other from various points of view.

### BRIEF DESCRIPTION OF THE DRAWINGS

FIG. 1 is a block diagram showing one example of an entire configuration of a matching system according to one embodiment of the present disclosure;

FIG. 2 is a block diagram showing one example of the configuration of a matching device according to one embodiment of the present disclosure;

FIG. 3 is a table showing one example of a data structure of an acquired information database updated by the matching device according to one embodiment of the present disclosure;

FIG. 4 is a table showing one example of a data structure of a classification result database updated by the matching device according to one embodiment of the present disclosure;

FIG. 5 is a flowchart for describing the flow of classification processing executed by the matching device according to one embodiment of the present disclosure; and

FIG. 6 is a flowchart for describing the flow of matching processing executed by the matching device according to one embodiment of the present disclosure.

### DETAILED DESCRIPTION

Hereinafter, one exemplary embodiment of the present disclosure will be described with reference to the attached drawings.

### [System Configuration]

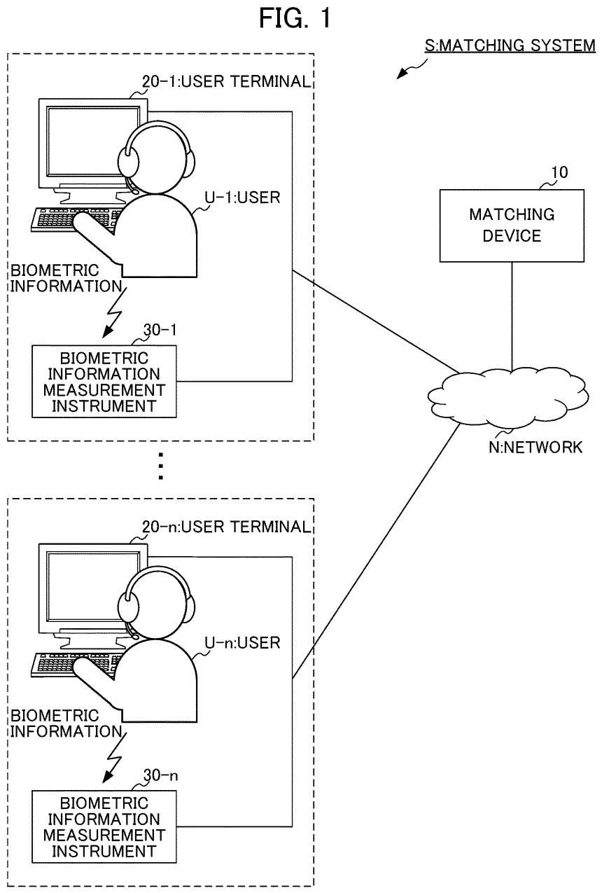

FIG. 1 is a block diagram showing an entire configuration of a matching system S according to the present embodiment. As shown in FIG. 1, the matching system S includes a matching device 10, a plurality of user terminals 20 (in the figure, a user terminal 20-1, . . . , a user terminal 20-n as one example), and a plurality of biometric information measurement instruments 30 (in the figure, a biometric information measurement instrument 30-1, . . . , a biometric information measurement instrument 30-n as one example). Moreover, FIG. 1 shows a plurality of users U (in the figure, a user U-1, . . . , a user U-n as one example) as subject persons targeted for processing performed by the matching system S.

A character “n” at the end of a reference numeral shown in FIG. 1 is an optional integer value of equal to or greater than one. Moreover, the values of n in FIG. 1 may be different values. That is, the number of devices included in the matching system S and the number of users utilizing the matching system S are not particularly limited. Further, the figure shows the matching device 10 as a single device, but a plurality of matching devices 10 may be present.

Note that in a case where description is made below without these n devices or n users being distinguished from each other, part of the reference numerals is omitted and these devices and users will be merely referred to as a “matching device(es) 10”, a “user terminal(s) 20”, a “biometric information measurement instrument(s) 30”, and a “user(s) U”.

These devices included in the matching system S are communicably connected to each other via a network N in the figure. Such communication among the devices may be performed in accordance with an optional communication method, and the communication method is not particularly limited. Moreover, communication connection may be wireless connection or wired connection. Further, communication may be directly performed among the devices without the network N. The network N includes, for example, a network such as a local area network (LAN), the Internet, and a mobile telephone network and a combination thereof.

The matching device 10 is a device to be utilized by a business operator (hereinafter simply referred to as a “business operator”) operating the matching system S. The matching device 10 is placed at, e.g., a store managed by the business operator or a server room managed by the business operator. On this point, the business category of the store managed by the business operator is not particularly limited. For example, the store may include a store such as a marriage agency providing opportunities for users to meet other users for the purpose of marriage, a store selling products, and a store mediating in the buying and selling of real estate.

The user terminal 20 and the biometric information measurement instrument 30 are devices utilized by the user U. The user terminal 20 and the biometric information measurement instrument 30 are, for example, placed at a user's home or the store managed by the business operator. In the case of placing these devices at the user's home, the user U possesses the user terminal 20 and the biometric information measurement instrument 30. Alternatively, the user U rents or leases the user terminal 20 and the biometric information measurement instrument 30 from the business operator. In this case, possession and rental/lease may be combined. For example, a form may be employed, in which the user U possesses the user terminal 20, but rents or leases the biometric information measurement instrument 30.

The matching system S having the above-described configuration asks a subject person (i.e., a certain user U targeted for matching) a question for matching the subject person with other persons (i.e., other users U as candidates for matching with the certain user U), thereby acquiring a response to the question from the subject person. Moreover, the matching system S acquires biometric information on the subject person. Further, the matching system S determines the state of the subject person based on the acquired biometric information. In addition, the matching system S classifies the subject person for matching based on the acquired response and the determined state of the subject person. Then, the matching system S matches the subject person and the other persons with each other based on a classification result obtained by a classification unit.

As described above, the matching system S makes a match not only based on the biometric information but also on the response to the question for matching. Thus, according to the matching system S, a match can be more appropriately made among the users from various points of view.

The business operator operates such a matching system S, thereby making a match among the users U for various purposes. For example, the business operator makes a match among users U seeking a personal relationship. In addition, the business operator makes a match between a user U as a provider providing a predetermined service and a user U as a user utilizing the predetermined service, for example. That is, the matching system S can be utilized for various purposes regardless of application. Hereinafter, it is, as one example for description, assumed that the business operator makes, by the matching system S, a match among users U seeking a personal relationship for a predetermined purpose. Although not particularly limited, the predetermined purpose may include purposes for dating, marriage, and pursuing hobbies together, for example.

Note that specific services according to application after the users have met each other according to matching by the matching system S are well-known by those skilled in the art. For this reason, the unique processing of the matching system S will be particularly described in detail below, and the details of the services after the users have met each other will not be described.

Next, each device included in the matching system S will be described in detail. The user terminal 20 provides the question for matching to the user U, and receives the response to the question from the user U. The user terminal 20 may include, for example, electronic equipment such as a personal computer, a tablet-type terminal, and a smartphone.

Specifically, the user terminal 20 receives, from the matching device 10, the question to be provided to the user U, thereby providing the question to the user U. For example, the question is provided in such a manner that the contents thereof are displayed on a display or a touch panel.

Moreover, the user terminal 20 receives the response to the question from the user U. For example, the response is received by operation of a keyboard and a mouse or a touch panel by the user U. Then, the user terminal 20 transmits, together with a user identifier for identifying the user U, the response received from the user U to the matching device 10. It may only be required that the user identifier is an identifier specific (i.e., unique) to each user U, and the type of information on the identifier is not particularly limited. Moreover, the above-described transmission may be performed by way of the biometric information measurement instrument 30.

The biometric information measurement instrument 30 measures the biometric information on the user U upon a response to the question for matching. The biometric information measurement instrument 30 uses, as a measurement method, a brain wave sensor, a line-of-sight sensor, an acceleration sensor, an electrocardiographic sensor, a Doppler sensor, or a combination thereof, thereby measuring the biometric information on the user U.

For example, in the case of utilizing the brain wave sensor, the biometric information measurement instrument 30 measures a brain wave change by means of a headphone-type brain wave sensor electrically contacting a body at two locations which are the forehead and earlobe of the user U. In the case of utilizing the electrocardiographic sensor, the biometric information measurement instrument 30 measures a change in a heart rate as one type of biometric information on the user U by means of a two-point-contact type electrocardiographic sensor configured such that electrodes are each touched by both thumbs of the user U.

In the case of utilizing the line-of-sight sensor, the biometric information measurement instrument 30 estimates the line of sight and the presence or absence of a blink by means of the line-of-sight sensor configured to measure electricity generated when a muscle is moved by an electrode contacting a facial skin (e.g., in the vicinity of nose pads of eye glasses). In the case of utilizing the acceleration sensor, the biometric information measurement instrument 30 observes small motion of the body by means of the acceleration sensor arranged on any location of a body trunk.

As the characteristics of each sensor, the brain wave sensor, the line-of-sight sensor, and the acceleration sensor have, for example, such characteristics that these sensors are suitable for measuring an instantaneous change due to a higher response speed than those of other sensors. On the other hand, the other sensors require a data collection time of about 10 seconds to one minute.

The Doppler sensor has such characteristics that such a sensor can measure information regarding the heart rate, a respiration rate, and body motion without contacting the body of the user U. For example, the Doppler sensor can measure the respiration rate, a ratio between an inhale time and an exhale time, and the depth of chest movement during respiration. On the other hand, the electrocardiographic sensor needs to contact the body of the user U for measurement. Note that the electrocardiographic sensor has, for example, such characteristics that such a sensor can measure a heart rate change with a higher accuracy than that of the Doppler sensor.

The business operator determines, as necessary, a biosensor to be used by the biometric information measurement instrument 30 based on the characteristics of each sensor and the type of biometric information necessary for matching. Note that these sensors may be used alone or in combination.

The biometric information measurement instrument 30 links, in chronological order, the brain wave change, the heart rate change, the line of sight/the presence or absence of a blink, or the small motion of the body measured by these biosensors with a measurement time, thereby generating the biometric information on the user U. Then, the biometric information measurement instrument 30 transmits, together with the user identifier for identifying the user U, the generated biometric information on the user U to the matching device 10. As such a user identifier, the same user identifier as that used by the user terminal 20 is utilized. Moreover, the above-described transmission may be performed by way of the user terminal 20.

The matching device 10 makes a match among the users U. The matching device 10 may include electronic equipment such as a server device and personal computer. Specifically, the matching device 10 receives and acquires the response from the user U, the response being transmitted from the user terminal 20. Moreover, the matching device 10 receives and acquires the biometric information on the user U, the biometric information being transmitted from the biometric information measurement instrument 30. Further, the matching device 10 makes a determination based on the acquired biometric information on the user U, thereby specifying the state of the user U upon a response to the question. In addition, the matching device 10 classifies, for matching, the user U based on the acquired response and the determined state of the subject person.

Then, the matching device 10 makes a match based on the classification result according to requests from matching applicants (in this case, the users U seeking the personal relationship for the predetermined purpose as described above) having applied for matching. Then, the matching device 10 provides a matching result to the matching applicants in the form of, e.g., a list.

The devices cooperate with each other as described above, and therefore, the matching system S functions as a system capable of making a match based on appropriate classification.

Each device included in the matching system S has been described above. Note that the device configuration shown in the figure is merely an example and the matching system S may have other device configurations. For example, the user terminal 20 and the biometric information measurement instrument 30 are not necessarily provided as separate devices, and may be provided as an integrated device. In this case, the matching device 10, the user terminal 20, and the biometric information measurement instrument 30 may be provided as an integrated device.

Alternatively, as shown in the figure, the plural pairs of the user terminal 20 and the biometric information measurement instrument 30 may be provided corresponding to the plurality of users U, and the single matching device 10 may collectively perform each type of processing for these plural pairs.

### [Configuration of Matching Device]

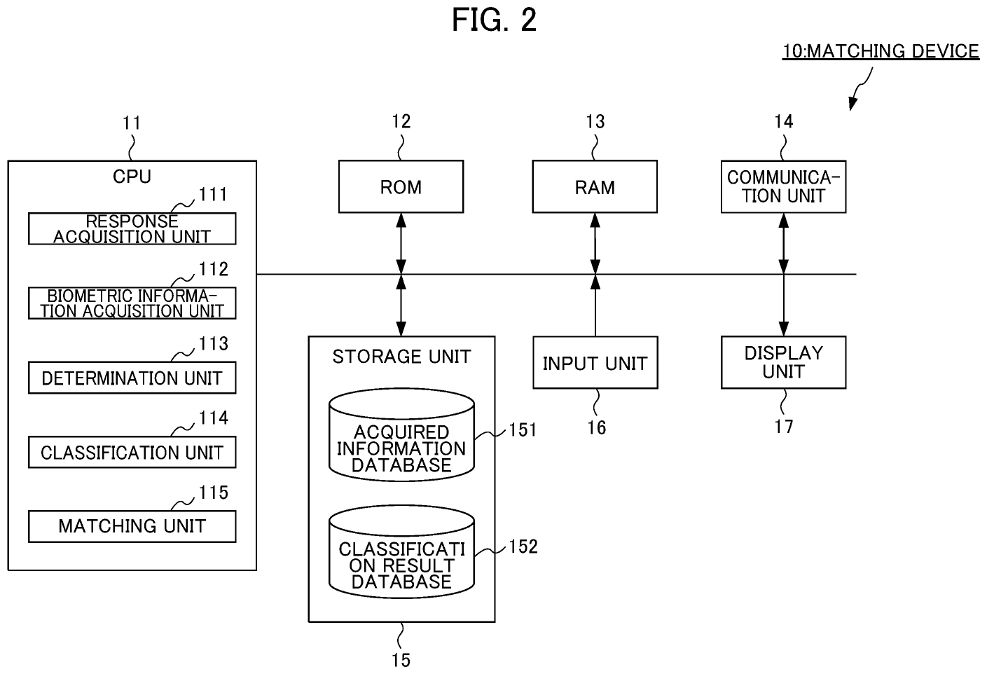

Next, the configuration of the matching device 10 will be described with reference to a block diagram of FIG. 2. As shown in FIG. 2, the matching device 10 includes a central processing unit (CPU) 11, a read only memory (ROM) 12, a random access memory (RAM) 13, a communication unit 14, a storage unit 15, an input unit 16, and a display unit 17. These units are bus-connected to each other via a signal line, thereby exchanging a signal.

The CPU 11 executes various types of processing according to a program recorded in the ROM 12 or a program loaded into the RAM 13 from the storage unit 15. The RAM 13 also stores, as necessary, data etc. necessary for execution of various types of processing by the CPU 11.

The communication unit 14 performs communication control for communication of the CPU 11 with other devices (e.g., the user terminal 20 and the biometric information measurement instrument 30). The storage unit 15 includes a semiconductor memory such as a dynamic random access memory (DRAM), and stores various types of data.

The input unit 16 includes various buttons and a touch panel or an external input device such as a mouse and a keyboard, and inputs various types of information according to user's instruction operation. The display unit 17 includes a display etc., and displays an image corresponding to image data output from the CPU 11.

In the matching device 10, these units cooperate with each other to perform “classification processing” and “matching processing”. The classification processing described herein is a series of processing in which the matching device 10 performs classification based on the response from the user U and the state of the user U determined from the biometric information on the user U to more appropriately classify the user from various points of view. The matching processing is a series of processing in which the matching device 10 makes a match based on an appropriate classification result obtained by the classification processing to provide a matching result to the matching applicant.

In the case of executing each of these types of processing, a response acquisition unit 111, a biometric information acquisition unit 112, a determination unit 113, a classification unit 114, and a matching unit 115 function in the CPU 11 as shown in FIG. 2. In a partial area of the storage unit 15, an acquired information database 151 and a classification result database 152 are stored. Including cases not specifically described below, data necessary for implementing each type of processing is, as necessary, exchanged among these functional blocks at appropriate timing.

The response acquisition unit 111 receives and acquires the response from the user U, the response being transmitted from the user terminal 20. Then, the response acquisition unit 111 stores the acquired response from the user U in the acquired information database 151. As a precondition to such storage, the response acquisition unit 111 stores the question to the user U in, e.g., the storage unit 15, and transmits the question to the user U to the user terminal 20.

The biometric information acquisition unit 112 receives and acquires the biometric information on the user U, the biometric information being transmitted from the biometric information measurement instrument 30. Then, the biometric information acquisition unit 112 stores the acquired biometric information on the user U in the acquired information database 151.

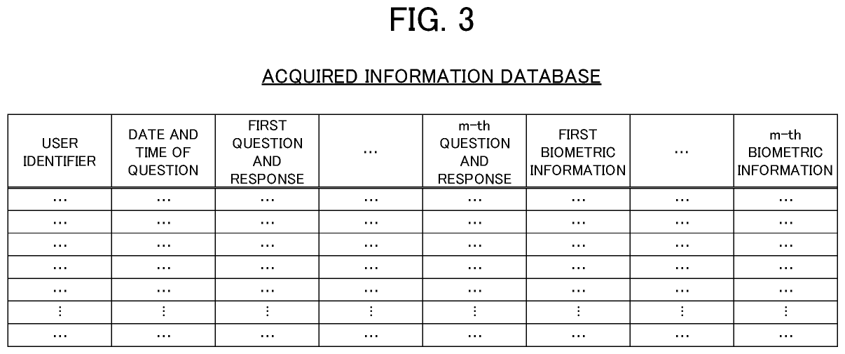

The acquired information database 151 described herein is a database storing various types of information used for performing the classification processing by the matching device 10. One example of a specific data structure of the acquired information database 151 will be described with reference to FIG. 3.

As shown in FIG. 3, the acquired information database 151 stores, as a single record, the user identifier in association with various types of information. For example, information corresponding to a single question group including continuous m (m is an optional integer value of equal to or greater than one) questions is stored as a single record.

In some cases, a plurality of records is stored for the same user U. For example, in a case where questions are provided to the same user U by means of a plurality of question groups including different question contents, information corresponding to each question group is stored as each record. Alternatively, in a case where questions are provided to the same user U multiple times by means of the same question group under different situations (e.g., the location and time for providing the questions) for providing the questions, information corresponding to each provision is stored as each record.

For example, each record includes columns from “USER IDENTIFIER”, “DATE AND TIME OF QUESTION”, and “FIRST QUESTION AND RESPONSE” to “m-th QUESTION AND RESPONSE” and columns from “FIRST BIOMETRIC INFORMATION” to “m-th BIOMETRIC INFORMATION”. Specific information contents corresponding to each column will be described.

“USER IDENTIFIER” is the identifier for identifying the user U corresponding to each record, and the same user identifier as those utilized by the user terminal 20 and the biometric information measurement instrument 30 is used. As described above, it may only be required that the user identifier is the identifier unique to each user U, and may be optional information. For example, an ID (identifier) assigned based on a predetermined rule may be taken as the user identifier.

“DATE AND TIME OF QUESTION” is information indicating the date and time of providing the questions corresponding to each record. For example, “DATE AND TIME OF QUESTION” is information indicating the period of time until provision of continuous m questions of a single question group (and acquisition of responses to these questions and the biometric information) ends after such provision has started.

The columns from “FIRST QUESTION AND RESPONSE” to “m-th QUESTION AND RESPONSE” indicate pairs of the matching question provided by the user terminal 20 and the response received by the user terminal 20. The question contents and the response method are not particularly limited, and various contents and methods can be selected according to application of the present embodiment. For example, the question may be a questionnaire for grasping the characteristics or preferences of the user U, and the response method may be selection from preset options by the user U.

The question includes, for example, questions necessary for specifying the character of the user U for character classification by the later-described classification unit 114. The character classification is, for example, classification based on a nationality, a gender, an age, a personality assessment result, and a psychopath diagnosis result.

On this point, personality assessment is, by the classification unit 114, made based on a personality test using the enneagram, a blood type, a constellation, or the synthetic personality inventory (SPI), for example. Thus, in the case of making the personality assessment, the question includes questions for making such personality assessment.

Further, the question includes, for example, questions necessary for specifying information necessary for contact between matched subject persons. In this case, the information necessary for contact includes, for example, a name, a gender, an address, a mail address, a telephone number, and information necessary for money transaction.

Further, the question may include questions necessary for specifying items emphasized by the user U in matching. This makes it possible, for example, to match a wide range of emphasized items.

For ensuring the credibility of responses to these various questions, the question may include, for example, questions for identification of the user U. In this case, the user U may be identified by a method such as a method working with a service (e.g., Facebook (registered trademark)) requiring utilization with a real name, for example.

The columns from “FIRST BIOMETRIC INFORMATION” to “m-th BIOMETRIC INFORMATION” indicate the biometric information measured by the biometric information measurement instrument 30, and is stored in association with the questions. That is, a first question and biometric information measured upon a response to such a question form first biometric information. Note that the biometric information to be stored includes, for example, the brain wave change, the heart rate change, the line of sight/the presence or absence of a blink, and small motion of the body, for example. The acquired information database 151 as described above is updated by the response acquisition unit 111 and the biometric information acquisition unit 112 every time the question to the user U is provided and the response to such a question is made from the user U.

The determination unit 113 makes determination based on the biometric information, which is stored in the acquired information database 151 by the biometric information acquisition unit 112, on the user U, thereby specifying the state of the user U upon a response to the question. The state includes, for example, comfort, impression, emotions, and mood, and one suitable for a purpose for classification is targeted for determination. For example, in the case of making determination based on the brain wave obtained as the biometric information on the user U, the brain wave is frequency-resolved by Fourier transform. Based on a frequency resolution result and the following criteria such as <Criteria for Determination based on Frequency>, the state of the user U can be determined.

<Criteria for Determination based on Frequency>

Theta (4 to 7 Hz):

at a high θ-wave ratio, a deep relaxed state and a light sleep state;
Alpha Low (8 to 9 Hz): a state in which the user U is not conscious of the outside world, and at a high α-wave ratio, a state in which the user U is calm both physically and mentally;
Alpha High (10 to 12 Hz): an open awareness state, and a state in which the user U can respond to a wide variety of status changes;
Beta Low (13 to 17 Hz): a problem-solved state,
at a high β-wave ratio, an active thinking/concentration state, and
a state in which the user U is nervous or has a certain amount of stress;
Beta High (18 to 30 Hz): a state relating to the strength of emotion (including both positive and negative);

### Gamma Low (31 to 40 Hz):

at a high γ-wave ratio, a state strongly relating to perception and consciousness, particularly a state with a higher mental activity (associated with plural matters), and
a state in which the user U feels strong anxiety or gets excited (not always negative).

Note that <Criteria for Determination based on Frequency> above are one example for determination and determination may be made based on other criteria or other criterion combinations. For example, it is, based on the heart rate, determined whether or not the parasympathetic nervous system is dominant. In this case, in a case where a frequency component (e.g., from 0.20 Hz to 0.15 Hz) in a high frequency band upon power spectrum analysis of a frequency component of a cardiac cycle change is greater than other frequency components, it can be determined that the parasympathetic nervous system is dominant A state in which many α-waves are confirmed as brain waves and the parasympathetic nervous system is dominant may be determined as a state of high comfort.

For example, in the case of making determination based on the line of sight and the presence or absence of a blink estimated by the line-of-sight sensor, a state such as the degree of concentration of the user U or the degree of sleepiness of the user U can be determined based on the line of sight and the presence or absence of a blink. For example, in a case where the line of sight is toward the left side of the user U, it can be determined that the user is looking back on one's past for making a response. In a case where the line of sight is toward the right side of the user U, it can be determined that the user U makes a response based on imagination. For example, in a case where the line of sight is toward the upper side of the user U, it can be determined that the user U makes a response based on information obtained from the eyes. In a case where the line of sight is toward the lower side of the user U, it can be determined that the user U makes a response based on information obtained from the nose, taste, or touch.

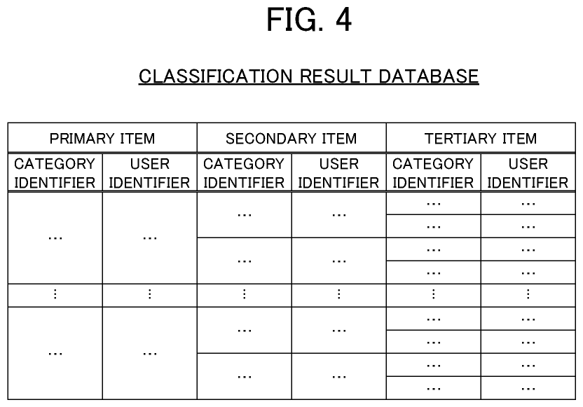

The classification unit 114 classifies (in this case, the character classification as described above) the user U based on the response, which is stored in the acquired information database 151 by the response acquisition unit 111, from the user U and the state, which is determined by the determination unit 113, of the user U upon a response to the question. Then, the classification unit 114 stores a classification result in the classification result database 152. As a precondition to such classification by the classification unit 114, the classification result database 152 will be first described. The classification result database 152 described herein is a database storing the classification result obtained by the classification unit 114. One example of a specific data structure of the classification result database 152 will be described with reference to FIG. 4.

As shown in FIG. 4, the classification result database 152 includes, as one example of categories for classifying the user U, categories each corresponding to three items including “Primary Item”, “Secondary Item”, and “Tertiary Item”. Upon classification, the user U is first classified as the category for the primary item. Moreover, the user U is also classified as the category for the secondary item, i.e., a detailed item of the primary item. Further, the user U is also classified as the category for the tertiary item, i.e., a further-detailed item of the secondary item. As described above, the categories for classifying the user U are provided in a hierarchical manner such that each category is detailed along the hierarchy. Each item includes, as columns, “Category Identifier” and “User Identifier”, for example. Specific contents of information corresponding to each column will be described.

“Category Identifier” is an identifier for identifying each category. It may only be required that the category identifier is, as in the user identifier, an identifier specific (i.e., unique) to each category, and may be optional information. For example, a name indicating the characteristic of the category may be taken as the category identifier.

“User Identifier” is the identifier of the user U classified as each item by the classification unit 114. The identifier itself used as the user identifier is the same as that used as the user identifier in the acquired information database 151. Such a classification result database 152 is updated by the classification unit 114 every time the question is provided to the user U, the response to such a question is obtained from the user U, and the biometric information is measured accordingly.

As a specific soring method, the classification unit 114 first weights a response to a corresponding question from the user U based on the state, which is determined by the determination unit 113, of the user U upon a response to the question. For example, the classification unit 114 weights a response to the first question based on the state, which is determined based on the first biometric information, of the user U upon a response to the first question. For example, in a case where the determined state of the user U upon a response to the question is the deep relaxed state or the physically-and-mentally calm state or a case where the user U is identified, it is assumed that the user U makes a response without hesitation, makes a response sincerely, or responds with a fact based on a previous event. That is, it is assumed that the credibility of such a response is high. For this reason, the classification unit 114 places a greater weight on such a response such that influence of the response on classification increases.

On the other hand, in a case where the determined state of the user U upon a response to the question is an unrelaxed state, a tension state, or a state with a certain amount of stress or a case where the line of sight is toward the right side of the user U as described above, it is assumed that the user U makes a response while hesitating, does not make a response sincerely, or makes a false response. That is, it is assumed that the credibility of such a response is low. For this reason, the classification unit 114 places less weight on such a response such that influence of the response on classification decreases.

The classification unit 114 increases, based on the responses weighted as described above, the influence of the response with a greater weight, and classifies the user U into the category of each of the primary, secondary, and tertiary items. Note that in a case where the classification unit 114 performs classification based on information corresponding to a single question group including continuous m questions and information corresponding to another question group for the same user is subsequently added, the classification unit 114 performs classification again based on the information on all question groups provided to the user U so far. In this case, a greater weight may be placed on a latest question group such that influence of information on the latest question group becomes greater.

As described above, the matching device 10 performs matching not only based on the biometric information but also on the response to the question for matching. Thus, according to the matching device 10, a match among the users can be more appropriately made from various points of view.

The matching unit 115 makes a match based on the classification result obtained by the 1140 according to requests from matching applicants (in this case, the users U seeking the personal relationship for the predetermined purpose as described above) having applied for matching. Then, the matching device 10 provides a matching result to the matching applicants in the form of, e.g., a list. Hereinafter, it is, as one example for description, assumed that the list of plural users U (hereinafter referred to as “matching partners”) having matched with a certain matching applicant is provided.

The matching unit 115 first receives a matching request from the matching applicant via operation by the input unit 16 or communication from other devices (e.g., any of the user terminals 20) via the communication unit 14. In this case, the matching unit 115 receives, for example, the attributes of candidates as the matching partners, the attributes being selected by the matching applicant. The attributes may include, for example, the gender, age, etc. of the candidate as the matching partner and direct indication of the classification of the candidate as the matching partner. Alternatively, a match may be made based only on the classification without selection of the attributes.

The matching unit 115 determines the candidates, i.e., the matching partners, to be in the list based on the selected attributes. In this case, the matching unit 115 does not necessarily include only the matching partners corresponding to the selected attributes themselves in the list, and may also include the matching partners corresponding to a certain range of the selected attributes in the list. For example, the range may be set to include attributes similar to the selected attributes, and the matching partners corresponding to such a range of the attributes may be included in the list.

Note that the attributes of each user U are obtained in such a manner that the user U responds to questions necessary for specifying the attributes as some of questions necessary for specifying the character of the user U, for example. For example, the gender, age, etc. of the user U are obtained in such a manner that the user U responses to questions for asking the gender, the age, etc. Alternatively, attributes input upon membership registration at, e.g., a store may be, as another method, also used with user U's consent.

The matching unit 115 first extracts, from the sorting result database 152, users U to be the candidates included in the list based on the selected attributes. Then, the matching unit 115 makes a match based on the extracted classification results (i.e., the classification categories) for the users U. Note that as described above, a match is made without such extraction in the case of making a match based only on the classification without selection of the attributes.

As a matching method, the matching unit 115 specifies, for example, the category into which the matching applicant oneself is classified, and as the matching partners, determines the users U of the same category as that of the matching applicant. Alternatively, the users U of the category compatible with that of the matching applicant are determined as the matching partners.

As the method for specifying the category of the matching applicant, the matching applicant may input the category of the matching applicant oneself when requesting matching. For example, in a case where, e.g., a sales representative which is a person engaging in a predetermined task for the matching partner requests matching, the category of the matching applicant may be specified by the above-described method. Alternatively, in a case where the matching applicant oneself is also one of the users U, the category of the matching applicant may be specified based on the result of the classification processing by the matching device 10. For example, in a case where a match is made among, e.g., members (each equivalent to the users U) of the marriage agency, the category of the matching applicant may be specified by the above-described method.

The matching unit 115 may not only make a match, but also may calculate an index value (hereinafter referred to as an “appropriate matching value”) indicating the appropriateness of matching. In this case, the matching unit 115 sets, for example, a high matching index value for a matching partner matched with the same category (or the category compatible with that of the matching applicant) as that of the matching applicant and also matched with the matching applicant in terms of the category of the tertiary item. Moreover, the matching unit 115 sets a lower matching index value for a matching partner not matched with the matching applicant in terms of the category of the tertiary item, but matched with the matching applicant in terms of the category of the secondary item. Further, the matching unit 115 sets a lowest matching index value for a matching partner not matched with the matching applicant in terms of the categories of the secondary and tertiary items, but matched with the matching applicant only in terms of the category of the primary item.

Then, the matching unit 115 forms the list of the matching partners determined as described above and the matching index value of each matching partner, thereby providing, as a matching list, such a list to the matching applicant. For example, the list is provided in such a manner that the matching list is displayed on the display unit 17 or the matching list is printed on paper. Note that the matching unit 115 may transmit information etc. on the matching applicant to the user terminals 20 used by the users U as the matching partners.

In this case, the matching unit 115 may further provide recommendation information on contact of the matching partner with the matching applicant to each matching partner included in the matching list. For example, the recommendation information may be information for establishing a smoother relationship with the matching partner, such as a particular keyword (e.g., what to say and what not to say) based on the attributes of the matching partner or contact timing (e.g., the timing of using the particular keyword).

The matching applicant can not only know about the matching partner, but also can obtain the appropriate matching value indicating the appropriateness of matching with the matching partner and the recommendation information on contact with the matching partner with reference to the above-described provided information. Accordingly, not only can a match be made based on an appropriate classification by the classification processing, but also information useful for selecting a final matching partner by the matching applicant and establishing a smooth relationship with the matching partner can be provided.

### [Classification Processing]

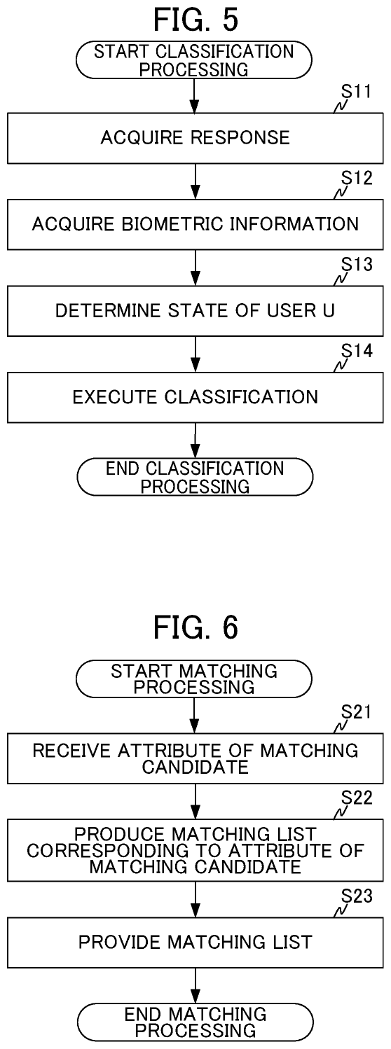

Next, the flow of the classification processing executed by the matching device 10 will be described with reference to a flowchart of FIG. 5. The classification processing is executed with the start of the question to the user U.

As Step S11, the response acquisition unit 111 acquires the response from the user U. At Step S12, the biometric information acquisition unit 112 acquires the biometric information on the user U. At Step S13, the determination unit 113 determines the state of the user U. At Step S14, the classification unit 114 executes classification. Then, the present processing ends.

### [Matching Processing]

Next, the flow of the matching processing executed by the matching device 10 will be described with reference to a flowchart of FIG. 6. The matching processing is executed with matching request operation by the matching applicant.

At Step S21, the matching unit 115 receives the attributes of the matching candidates from the matching applicant. At Step S22, the matching unit 115 produces the matching list corresponding to the attributes of the matching candidates received at Step S21. At Step S23, the matching unit 115 provides the matching list. Then, the present processing ends.

According to the classification processing and the matching processing as described above, a match is made not only based on the biometric information but also on the response to the question for matching. Thus, according to the matching system S, a match among the users can be more appropriately made from various points of view. Moreover, not only can a match be made based on the appropriate classification, but also the information useful for the matching applicant, such as the matching index value for each matching partner and the recommendation information on contact with the matching partner, can be provided.

### Application Examples

In the above-described embodiment, a match is made among the users U seeking the personal relationship for the predetermined purpose. The present disclosure is not limited to above, and the matching system S can be utilized for various purposes regardless of application. For example, the matching system S can be utilized for purposes such as the following application examples.

### <Application Examples Related to Provision and Utilization of Predetermined Service>

As a specific application example, the matching system S can make a match between a user U as a provider providing a predetermined service and a user U as a user utilizing the predetermined service. Although not particularly limited, the contents of the predetermined service include, for example, a delivery request service between individuals, a language teaching service between individuals, and a service for mediating in the buying and selling of goods between individuals.

Note that the method for providing the predetermined service after matching by the matching system S is well-known by those skilled in the art, and for this reason, the processing unique to the matching system S will be described in detail below and description of the service providing method itself will be omitted as necessary.

In the present application example, in a case where the user U is the provider of the service, the information used for matching by the matching unit 115 includes the contents (e.g., a provided service item, time and location that the service can be provided, a cost for providing the service) of the service provided to the user U, a past service record (e.g., the number of services provided, a past sales record, and evaluation from the partner having received the service) for the user U, and the character of the user U. In this case, the question for classification by the classification unit 114 includes questions for specifying the provided service contents and the past service record. The classification unit 114 performs classification, including the provided service contents and the past service record. Alternatively, as another method, the classification unit 114 may perform only the character classification without considering the provided service contents and the past service record, and the provided service contents and the past service record may be stored in association with the character classification result.

On the other hand, in a case where the user U is the user of the service, the information used for matching by the matching unit 115 includes the contents (e.g., a used service item, time and location that the user U wishes to utilize the service, and a cost for receiving the service) of the service utilized by the user U, a past request record (e.g., the number of services requested, a past payment record, and evaluation from the partner having provided the service) for the user U, and the character of the user U. In this case, the question for classification by the classification unit 114 includes questions for specifying the used service contents and the past request record. The classification unit 114 performs classification, including the used service contents and the past request record. Alternatively, as another method, the classification unit 114 may perform only the character classification without considering the used service contents and the past request record, and the used service contents and the past request record may be stored in association with the character classification result.

Then, the matching unit 115 makes a match between the user U as the service provider and the user U as the service user based on the classification result. Alternatively, in a case where the character classification result and the service contents etc. are stored in association with each other as in the above-described another method, the matching unit 115 makes a match between the user U as the service provider and the user U as the service user based on the classification result and the service contents etc. stored in association with the classification result.

In this manner, a match can be made not only based on the character of each user U but also on the provided service contents and the past service record on the side of the service provider and the used service contents and the past request record on the side of the service user. Thus, a match can be more suitably made for the purpose of providing and utilizing the predetermined service.

Note that in a case where the matching applicant is, for example, the user U as the service user, the user U may select, as the method for subsequently providing the predetermined service, an intended user U from a plurality of users U as service providers in the matching list, thereby confirming the request. Then, the user U selected as the service provider may check information (e.g., requested service contents, a past use record for the user U as the service user, and the character of the user U as the service user) on the confirmed request from the user U as the service user, thereby confirming a service order.

Conversely, in a case where the matching applicant is the user U as the service provider, the user U may select an intended user U from a plurality of users U as service users in the matching list, thereby confirming a service request. Then, the user U selected as the service user may check information (e.g., provided service contents, a past service record for the user U as the service provider, and the character of the user U as the service provider) on the confirmed request from the user U as the service provider, thereby confirming the request.

A structure may be provided, in which after a predetermined service corresponding to the request confirmed as described above has been provided, the user U as the service user and the user U as the service provider evaluate each other. It may be configured such that without such evaluation, the processing cannot proceed to the step of paying a fee for using the predetermined service. In this case, the matching unit 115 may also perform processing (e.g., the processing of managing communication between the users U) relating to the predetermined service.

Note that in description above, it is assumed that both the user U as the service user and the user U as the service provider respond to the question and are classified by the classification unit 114. The present disclosure is not limited to such a case, and for example, only the user U as the service provider may respond to the question and may be classified by the classification unit 114. In this case, when the user U as the service user requests matching as the matching applicant, such a user U inputs the used service contents, the past request record, and the character of the service provider as a request for the matching applicant. Then, a match may be made based on the input contents and the classification result for the user U as the service provider.

Note that various configurations of the above-described embodiment are also included as, e.g., the following application examples, needless to say. For example, in a case where the matching applicant is the user U as the service user, the matching unit 115 may provide the matching list of a plurality of matched users U as service providers to the matching applicant. Conversely, in a case where the matching applicant is the user U as the service provider, the matching unit 115 may provide the matching list of a plurality of matched users U as service users to the matching applicant. In this case, the matching list including the above-described appropriate matching values may be provided.

Further, in the case of the matching list of a plurality of users U as service providers, the matching list including the provided service contents, the past service record, and the character for each of the plurality of users U as the service providers may be provided. Conversely, in the case of the matching list of a plurality of users U as service users, the matching list including the used service contents, the past request record, and the character for each of the plurality of users U as the service users may be provided.

In addition, the question may include, for example, a question for asking which one of the provided service contents, the past service record, the used service contents, the past request record, or the character is emphasized. With this configuration, a range can be set for the emphasized item, and a match can be made for such a range, for example.

### <Application Examples Unrelated to Provision and Utilization of Predetermined Service>

As other specific application examples, the matching system S can make a match among the users U regardless of provision and utilization of the predetermined service by the users U.

In the present application example, the information used for matching by the matching unit 115 includes matching conditions (e.g., a gender, an age, a body type, a desired area, an available period of time, and a hobby) and the character. In this case, the question for classification by the classification unit 114 includes questions for specifying these matching conditions. The classification unit 114 performs classification, including these matching conditions. Alternatively, as another method, the classification unit 114 may perform only the character classification without considering the matching conditions, and the matching conditions may be stored in association with the character classification result.

Then, the matching unit 115 makes a match among the users U based on the classification result. Alternatively, as in a case where the character classification result and the matching conditions are stored in association with each other as in the above-described another method, the matching unit 115 makes a match among the users U based on the classification result and the matching conditions stored in association with the classification result.

With this configuration, a match can be made not only based on the character of each user U but also on the matching conditions for each user U. Thus, a match can be more suitably made for the purpose of making a match among the users U regardless of provision and utilization of the predetermined service by the users U.

Note that as a specific method until a match is established among the users U after matching, a user U (for the sake of convenience, referred to as a “first user U”) as a matching applicant may select candidate users U (for the sake of convenience, referred to as “second users U”) as candidates for matching from a plurality of users U in a matching list, thereby confirming the proposed candidates.

Meanwhile, the second users U proposed and confirmed as the candidates determines, with reference to information on the first user U having requested matching, whether or not the second users U request matching. A notification indicating that a match has been established is provided to the first and second users U both having requested matching.

In this case, the matching unit 115 may perform processing (e.g., the processing of managing communication among the users U) relating to the specific method until a match is established.

Note that various configurations of the above-described embodiment are also included as, e.g., the following application examples, needless to say. For example, the matching unit 115 may provide the matching list of a plurality of matched users U to the matching applicant.

Further, in the case of the matching list of a plurality of users U, the matching list including the matching conditions for each of the plurality of users U may be provided. In addition, the matching list including information on compatibility among the matching applicant and the plurality of users U may be provided. In this case, the matching list including both the matching conditions and the compatibility information may be provided. Further, the user U as the matching applicant may conduct a refined search from the matching list based on the provided matching conditions and compatibility information.

The compatibility information described herein may be based on the character classification, but a correlation among items obtained from past data analysis may be also utilized for the compatibility information. For example, in the case of hobby categories seemed to be unrelated to each other, if the data analysis finds that there are hobbies compatible with these categories, the compatibility information may be produced based on such an analysis result. In addition, if the data analysis finds, for example, that a user U who “likes a person who makes me (the user U) laugh” and a user U posting a picture of the user U smiling as one's profile are compatible with each other, the compatibility information may be produced based on such an analysis result. In addition, the compatibility information may be produced based on the action history of the subject person, such as a time period in which the user U utilizes the matching system S by means of the user terminal 20.

### [Variations]

The embodiment of the present disclosure has been described above, but this embodiment is merely an example and is not intended to limit the technical scope of the present disclosure. Various other embodiments may be included in the present disclosure, and various changes such as omissions and replacements may be made without departing from the gist of the present disclosure. These embodiments and variations thereof are included in the scope and gist of the present disclosure described in the present specification etc., and are included in the scope of the disclosure described in the claims and the equivalency thereof. For example, the embodiment of the present disclosure may be modified as in the following variations.

### <First Variation>

In the above-described embodiment, the matching unit 115 solely makes a match based on the classification result obtained by the classification unit 114. The present disclosure is not limited to above, and a match may be made considering other types of information. For example, a system for producing a community based on a common hobby, value, etc. by the users U is assumed. In this case, each user U can freely join the community.

In this situation, the matching unit 115 takes, when making a match, users U joining the same community as that for the matching applicant as compatible matching candidates on a preferential basis. For example, only users U joining the same community as that for the matching candidates are selected from the matched users U based on the classification result obtained by the classification unit 114, and are included in the matching list.

Alternatively, in a case where the matching list is produced based on the classification result obtained by the classification unit 114, users U joining the same community as that for the matching applicant are high on the matching list. Alternatively, the user U as the matching applicant can conduct a refined search from the matching list based on the community that each user U of the matching list joins.

With this configuration, a match can be made not only based on the classification result obtained by the classification unit 114 but also on information indicating the community based on the common hobby, value, etc. for each user U. Thus, in a case where the system for producing the community is present, a match can be more suitably made.

### <Second Variation>

In the above-described embodiment, the response acquisition unit 111 stores the question to the user U in, e.g., the storage unit 15, and transmits the question to the user U to the user terminal 20. Then, the user terminal 20 receives such a question, and provides the question to the user U. In this case, the response acquisition unit 111 may determine the contents of a new question to the user U based on the result obtained once by the classification processing.

For example, in a case where the user U is classified as a certain category, there is a probability that such a user U actually needs to be classified not as the certain category but as another category similar to the certain category. In this case, the contents of the new question to the user U may be such contents that the classification of the user U can be distinguished between the certain category and another category. As described above, the contents of the new question to the user U are determined based on the result obtained once by the classification processing, and therefore, classification can be performed with a higher accuracy.

### <Third Variation>

In the above-described embodiment, the matching unit 115 determines the matching partner based on the classification result obtained by the classification processing. The present disclosure is not limited to above, and the matching partner may be determined based on each type of information used for classification by the classification unit 114. For example, the matching partner may be determined using the response itself from the user U for classification by the classification unit 114 or the biometric information itself upon a response from the user U. For example, the matching unit 115 may select, as the matching partner, a user U providing the same response as that of the matching applicant or a user U having the same biometric information as that upon a response from the matching applicant. Alternatively, the matching unit 115 may increase the matching index values of these users U.

### <Fourth Variation>

In the above-described embodiment, the classification unit 114 performs classification based on the response from the user U and the state, which is determined from the biometric information upon a response from the user U, of the user U upon a response therefrom. The present disclosure is not limited to above, and classification may be performed based on the state of the user U determined from the biometric information at the point of time different from the time of a response.

For example, classification may be performed based on the state of the user U upon experiencing trial contents using virtual reality (VR), the state being determined from the biometric information upon experiencing the trial contents. In this case, the user U wears, e.g., a goggle-type device for providing the virtual reality. With the goggle-type device, the user U experiences the trial contents. As in the time of responding to the question, the biometric information measurement instrument 30 measures the biometric information on the user U. Based on such biometric information, the determination unit 113 determines the state of the user U.

Based on the state of the user U in the above-described unrealistic situation, the characteristics of the user U that the user U oneself does not notice can be revealed. Thus, the classification unit 114 can perform classification according to the characteristics of the user U.

Note that the present variation may be further modified to form the biometric information measurement instrument 30 by, e.g., the goggle-type device for providing the virtual reality. For example, a sensor may be incorporated into the goggle-type device to form the biometric information measurement instrument 30. With this configuration, the biometric information can be measured without the user U being aware of the attached sensor.

### [Implementation by Hardware, Software, etc.]

Each device included in the above-described embodiment is not limited to the form of the above-described embodiment, and may be general electronic equipment having an information processing function. A series of processing as described above may be executed by hardware or software. A single functional block may include hardware alone, software alone, or a combination thereof. In other words, the functional configuration shown in FIG. 2 is merely an example, and is not particularly limited. That is, it is enough that the matching system S has the function of executing a series of processing as described above as a whole, and use of the functional blocks for implementing such a function is not particularly limited to that in the example of FIG. 2.

For example, the functional configuration of the present embodiment may be implemented by a processor configured to execute arithmetic processing, and the processor available for the present embodiment includes various processing devices alone such as a single processor, a multi-processor, and a multi-core processor; and combinations of these various processing devices with processing circuits such as an application specific integrated circuit (ASIC) and a field-programmable gate array (FPGA).

In the case of executing a series of processing by the software, a program forming the software is installed on, e.g., a computer from a network or a recording medium. The computer may be a computer incorporated into dedicated hardware. The computer may be a computer, such as a general-purpose personal computer, capable of executing various functions by installation of various programs.

The recording medium including the program may be, for providing the program to a user, distributed to the user separately from a device body or may be provided to the user with the recording medium being incorporated into the device body in advance. The recording medium distributed separately from the device body includes, for example, a magnetic disk (including a floppy disk), an optical disk, and a magnetic optical disk. The optical disk includes, for example, a compact disc-read only memory (CD-ROM), a digital versatile disc (DVD), and a Blu-ray (the registered trademark) disc. The magnetic optical disk includes, for example, a mini disc (MD). The recording medium provided to the user with the recording medium being incorporated into the device body in advance includes, for example, the ROM 12 of FIG. 2 recording the program and a hard disk included in the storage unit 15 of FIG. 2.

Note that in the present specification, the steps of describing the program recorded in the recording medium include not only processing performed in a chronological order, but also processing not performed in a chronological order but executed in parallel or separately. Moreover, in the present specification, the term “system” means an entire device including a plurality of devices, a plurality of sections, etc.

What is claimed is:

1. An information processing device comprising:
a processor configured to
acquire a response from at least one subject person to a question provided to the subject person for matching the subject person with any other person;
acquire biometric information on the subject person;
determine a state of the subject person based on the biometric information acquired;
classify the subject person for matching based on the response acquired and the state of the subject person to generate a classification result; and
match the subject person and the other person with each other based on the classification result.

2. The information processing device according to claim 1, wherein
the question to the subject person for matching includes a question necessary for specifying a character of the subject person.

3. The information processing device according to claim 1, wherein
the question to the subject person for matching includes a question necessary for specifying information necessary for contact between the subject person and the other person to which the subject person is matched.

4. The information processing device according to claim 1, wherein
the question to the subject person for matching includes a question necessary for specifying an item emphasized for matching by the subject person.

5. The information processing device according to claim 1, wherein
the response to the question for matching is weighted as information used for classification based on the state of the subject person.

6. The information processing device according to claim 1, wherein
the biometric information acquired is biometric information measured from the subject person upon a response to the question for matching from the subject person.

7. The information processing device according to claim 1, wherein
the biometric information acquired is biometric information measured from the subject person when the subject person experiences trial contents.

8. The information processing device according to claim 1, wherein
the processor is configured to match a service provider as the subject person and a service user utilizing a service provided by the service provider.

9. The information processing device according to claim 8, wherein
in addition to the classification result, the processor is configured to make a match based on information regarding contents of the service provided by the service provider, information regarding a record of the service provided by the service provider, information regarding a character of the service provider, and a condition requested by the service user regarding the information.

10. The information processing device according to claim 8, wherein
the processor is configured to
provide, as service provider candidates for matching, a plurality of service providers to the service user, and
upon provision of the candidates, provide at least any of information regarding contents of a service provided by each of the plurality of service providers, information regarding a record of the service provided by each of the plurality of service providers, and information regarding a character of each of the plurality of service providers to the service user.

11. The information processing device according to claim 8, wherein
the service user is another subject person.

12. The information processing device according to claim 1, wherein
the processor is configured to match the subject person with the other person which is another subject person.

13. The information processing device according to claim 12, wherein
the processor is configured to make a match based on a condition required for a matching partner by the subject person in addition to the classification result.

14. The information processing device according to claim 12, wherein
the processor is configured to
provides, as subject person candidates for matching, a plurality of subject persons to other subject persons, and
upon provision of the subject person candidates, provides compatibility information on the subject persons based on the classification result to the other subject persons.

15. The information processing device according to claim 1, wherein
the subject person or persons join an optional community or communities, and
the processor is configured to make a match based on the community or communities that the subject person or persons join in addition to the classification result.

16. The information processing device according to claim 2, wherein
the question to the subject person for matching includes a question necessary for specifying information necessary for contact between the subject person and the other person to which the subject person is matched.

17. The information processing device according to claim 2, wherein
the question to the subject person for matching includes a question necessary for specifying an item emphasized for matching by the subject person.

18. The information processing device according to claim 2, wherein
the response to the question for matching is weighted as information used for classification based on the state of the subject person.

19. The information processing device according to claim 2, wherein
the biometric information acquired is biometric information measured from the subject person upon a response to the question for matching from the subject person.

20. A non-transitory computer-readable medium including an information processing program which causes a computer to implement:
a response acquisition function of acquiring a response from a subject person to a question provided to the subject person for matching the subject person with other persons,
a biometric information acquisition function of acquiring biometric information on the subject person,
a determination function of determining a state of the subject person based on the biometric information acquired by the biometric information acquisition function,
a classification function of classifying the subject person for matching based on the response acquired by the response acquisition function and the state of the subject person determined by the determination function, and
a matching function of matching the subject person and the other persons with each other based on a classification result obtained by the classification function.
